(19) United States
(12) Patent Application Publication
(10) Pub. No.: US 2018/0193267 A1
(43) Pub. Date: Jul. 12, 2018

(54) NANOLIPOSOMAL FORMULATION FOR TARGETING INTEGRIN-LINKED KINASE (ILK) WITH SMALL-INTERFERENCE RNA AND A METHOD FOR TREATING OVARIAN CANCER

(71) Applicant: Pablo E. Vivas-Mejia, San Juan, PR (US)

(72) Inventor: Pablo E. Vivas-Mejia, San Juan, PR (US)

(21) Appl. No.: 15/384,299

(22) Filed: Dec. 19, 2016

Related U.S. Application Data
(63) Continuation-in-part of application No. 14/681,875, filed on Apr. 8, 2015, now Pat. No. 9,895,312.
(60) Provisional application No. 62/268,792, filed on Dec. 17, 2015.

(51) Int. Cl. A61K 9/127; A61K 31/713; A61K 47/24; A61K 47/10; A61P 35/00; C12N 15/113

(52) U.S. Cl. CPC A61K 9/1271; A61K 31/713; C12N 15/1135; A61K 47/10; A61P 35/00; A61K 47/24

(57) ABSTRACT

The invention provides a nanoliposomal formulation that targets Integrin Linked Kinase (ILK) with small-interfering RNA (siRNA) in cisplatin-resistant ovarian cancer. A reduction in cell proliferation and invasion was observed upon ILK depletion by siRNA treatment. Furthermore, an ILK-targeted siRNA-liposomal formulation reduced tumor metastasis in an ovarian cancer mouse model. SiRNA-mediated ILK silencing reduced the phosphorylation levels of AKT and GSK3β. The nanoliposomal formulation is formed by lipids and ILK-siRNA according to specific ratios.

### GOVERNMENT INTEREST

The claimed invention was made with U.S. Government support under grant numbers R25-GM061838 (JMRG) and RTRN U54MD008149 awarded by National Institute of Health (NIH). The government has certain rights in this invention.

### BACKGROUND OF THE INVENTION

Ovarian cancer remains an important cause of female cancer-related deaths, with approximately 22,280 new cases and about 14,240 deaths predicted for 2016. Its high death rate is reflective of the fact that most ovarian cancer patients are diagnosed with advanced-stage disease (stages III and IV). The most common types of ovarian cancer include epithelial ovarian carcinoma (90% of all ovarian malignancies), germ-cell tumors and sex-cord stromal tumors. Reports indicate that epithelial ovarian cancer (EOC) is a complex group of diseases that based on histopathology and molecular genetic alterations could be divided into five main types, high-grade serous, endometrioid, clear-cell, mucinous, and low-grade serous carcinomas. High-grade serous ovarian cancer (HGSOC) represents 70% of all EOC. Despite good responses to first-line treatment with platinum-based combination chemotherapy, most HGSOC patients will relapse and eventually develop platinum-resistant disease with a poor overall prognosis. Therefore, new therapies targeting key survival pathways are urgently needed for this deadly disease.

ILK is a kinase and adaptor protein connecting the extracellular matrix (ECM), cell membrane-associated proteins (i.e. integrins), and cell signaling pathways. Increased levels of ILK have been detected in several cancers, and have been associated with cancer progression, epithelial-mesenchymal transition (EMT), angiogenesis and metastasis. ILK binds to beta integrin subunits and phosphorylates protein kinase B (AKT) leading to integrin-mediated cell proliferation and survival. Another ILK target is glycogen synthase kinase 3β (GSK3β), whose phosphorylation leads to stabilization and accumulation of beta-catenin (b-catenin) in the cytoplasm and its subsequent movement into the nucleus where b-catenin associates with transcription factors to activate expression of several genes, including c-Myc and Cyclin D1. Hwang and co-workers showed that tumor growth factor (TGFβ1) activates ILK and stimulates invasion and migration of the SKOV3 human ovarian cancer cells. Similarly, Cho and co-workers showed that ILK-targeted siRNA abrogated the invasive potential of ovarian cancer cells and reduced in vivo tumor growth. However, few are known about, the molecular and therapeutic biological effects of ILK silencing in cisplatin resistant ovarian cancer. Here we observed that the phosphorylation levels of ILK are higher in cisplatin resistant compared with cisplatin sensitive ovarian cancer cells. Furthermore, we studied the molecular and therapeutic effects of targeting ILK in cisplatin resistant ovarian cancer.

### BRIEF DESCRIPTION OF THE DRAWINGS

The detailed description may be better understood with reference to the accompanying figures that are provided for illustrative purposes and are not to be considered limiting any aspect of the invention, in which:

FIG. 1 shows protein expression of ILK downstream molecules in cancer cells and human tumors.

FIGS. 2A-2F show the effects of siRNA-mediated silencing of ILK in vitro, according to the present invention.

FIGS. 3A-3B show the effects of ILK silencing in combination with cisplatin on cell viability, according to the present invention.

FIG. 4A shows Western blot analysis indicting that nanoliposomal-ILK-siRNA reduced ILK protein levels in vivo, according to the present invention.

FIG. 4B shows average densitometry values indicating that the observed reduction in ILK protein levels was significant when compared to C-siRNA group, according to the present invention.

FIG. 4C shows Mean tumor weight when treated according to the present invention.

FIG. 4D shows number of tumor nodules recorded after the end of the treatment period, according to the present invention.

FIG. 5 shows Western blot analysis indicating that transient transfection in A2780CP20 cells with ILK-siRNA(1) and ILK-siRNA(2) significantly reduced ILK protein levels, according to the present invention.

FIG. 6 shows a schematic representation of a PEG-2000 nanoliposome, according to the present invention.

### DETAILED DESCRIPTION OF THE INVENTION

### Materials and Methods:

### Cells and Culture Conditions

The human ovarian epithelial cancer cells A2780CP20, HEYA8, and HEYA8.MDR were obtained and have been described elsewhere. A2780 cells were purchased from the European Collection of Cell Cultures (ECACC) and OV90 cells from American Type Culture Collection (ATCC). Cisplatin-resistant ovarian cancer OV90CIS cells were generated by exposing the parental OV90 cells to increasing concentrations of cisplatin. Cells were maintained in RPMI-1640 medium supplemented with 10% fetal bovine serum (FBS) and 0.1% antibiotic/antimycotic solution at 37° C. in 5% CO₂ and 95% air. All cell lines were screened for Mycoplasma using the LookOut® Mycoplama PCR detection kit (Sigma-Aldrich). In vitro assays were performed at 70-85% cell density.

### Western Blot Analysis

Cell pellets were washed with PBS and lysed with ice-cold lysis buffer (1% Triton X, 150 mM NaCl, 25 mM Tris HCl, 0.4 mM NaVO₄, 0.4 mM NaF, and protease inhibitor cocktail from Sigma), and total protein concentration was determined using Bio-Rad DC Protein Assay reagents (Bio-Rad, Hercules, Calif.) following the manufacturer's instructions. Protein samples (30-50 mg per well) were separated by SDS-PAGE and blotted onto nitrocellulose membranes. The membranes were blocked in either 5% non-fat dry milk or 5% BSA for 1 hour at room temperature, and incubated overnight at 4° C. with phospho-ILK (Millipore), ILK, phospho-AKT (Ser473), AKT, phospho-GSK3β (Ser9), and GSK3β (Cell Signaling) primary antibodies. For primary antibody detection, membranes were incubated 1 hour at room temperature with mouse or rabbit IgG horseradish peroxidase (HRP)-linked secondary antibodies. The bound antibodies were detected using enhanced chemiluminescence (GE Healthcare) followed by autoradiography. Bands were imaged with a FluorChem system (Alpha Innotech Corporation, San Leandro, Calif.) and the signal intensity of each band was quantified using AlphaEaseFC software. All membranes were reprobed with β-actin monoclonal antibody (Sigma) as a normalizing control.

Small Interfering RNA (siRNA) and In Vitro siRNA Transfection

To silence human ILK (NM_001014794.2) two siRNAs [5′-CTCAATAGCCGTAGTGTAA-3′ (siRNA-1), and 5′-GTCAAGTTCTCTTTCCAAT-3 (siRNA-2)] targeting different sites in the ILK mRNA, and a non-silencing negative control siRNA (C-siRNA) were used. All siRNAs were purchased from Sigma. Briefly, A2780CP20 and OV90CIS cells (2×10⁴ cells/mL or 3.5×10⁴ cells/mL) were seeded into 10 cm Petri dishes. Twenty-four hours later, siRNAs were mixed with HiPerfect transfection reagent (Qiagen) at 1:2 ratio (siRNA:transfection reagent) in serum and antibiotic-free Opti-MEM medium (Life Technologies), incubated 10 minutes at room temperature and then added to the cells. Transfected cells were incubated at 37° C. and collected 24 hours later for assessment of ILK protein levels by Western blot analysis.

### In Vitro Cell Viability and Cell Growth

For cell viability assays, A2780CP20 cells (2×10⁴ cells/mL) were seeded into 96-well plates. Twenty-four hours later, siRNAs were added to the cells, and the transfection mix was replaced with fresh media after 24 hours of treatment for additional 48 hours. To assess the effect of combination treatment with siRNA and cisplatin (CIS), cells were seeded as described above, and treated with siRNAs for 24 hours and with cisplatin for 48 hours. Seventy-two hours after siRNA transfection, cell viability was measured and calculated as described above. In all cases, the medium was removed at the end of the treatment and 95 μL of Alamar blue dye (Invitrogen) was added following the manufacturer's instructions. OD values were obtained spectrophotometrically in a plate reader (BioRad) after 3-4 hours of Alamar blue dye incubation. Percentages of cell viability were obtained after blank OD subtraction, taking the untreated cell values as 100% of cell viability.

For assessment of cell growth, colony formation assays were performed using crystal violet dye. Briefly, A2780CP20 and OV90CIS cells (3×10⁴ cells/mL or 4.5×10⁴ cells/mL) were seeded into 6-well plates. Twenty-four hours later, siRNAs were added to the cells and incubated at 37° C. Sixteen to eighteen hours after transfection, 1000 (A2780CP20) or 2,500 (OV90CIS) cells were seeded into 10 cm Petri dishes. Seven (A2780CP20) or ten (OV90Cis) days later, colonies were fixed and stained with 0.5% crystal violet solution in methanol. Colonies of at least 50 cells were scored in five random fields using a light microscope (Olympus CKX41) with a total magnification of 40×.

### In Vitro Cell Invasion

The effects of ILK silencing on cisplatin-resistant ovarian cancer cell invasion were analyzed using the transwell method with a matrigel. A2780CP20 and OV90CIS cells (3.5×10⁴ cells/mL) were seeded into 10 cm Petri dishes and transfected overnight with siRNAs as previously above described. The next day, the invasive ability of the transfected cells was evaluated in 24-well transwell plates (Corning Incorporated). Briefly, 60 mL of matrigel (BD Biosciences) in serum-free media was added onto the upper chambers and incubated for at least 1 hour at 37° C. for polymerization. Transfected cells were collected and resuspended in serum-free RPMI1640 medium and re-seeded onto the upper well of the matrigel-coated chamber. RPMI1640 medium containing 10% FBS was added to the lower wells and the chambers were incubated at 37° C. After 48 hours in the transwell, media was removed and cells that invaded through the matrigel were fixed and stained using the Protocol Hema 3 Stain Set (Fisher Scientific) as previously described. The invading cells were counted in five randomly selected fields using a light microscope with a total magnification of 40×. Percentages of cell invasion were calculated taking the C-siRNA transfected cell values as 100% of cell invasion.

SiRNA Incorporation into DOPC-PEG-Nanoliposomes

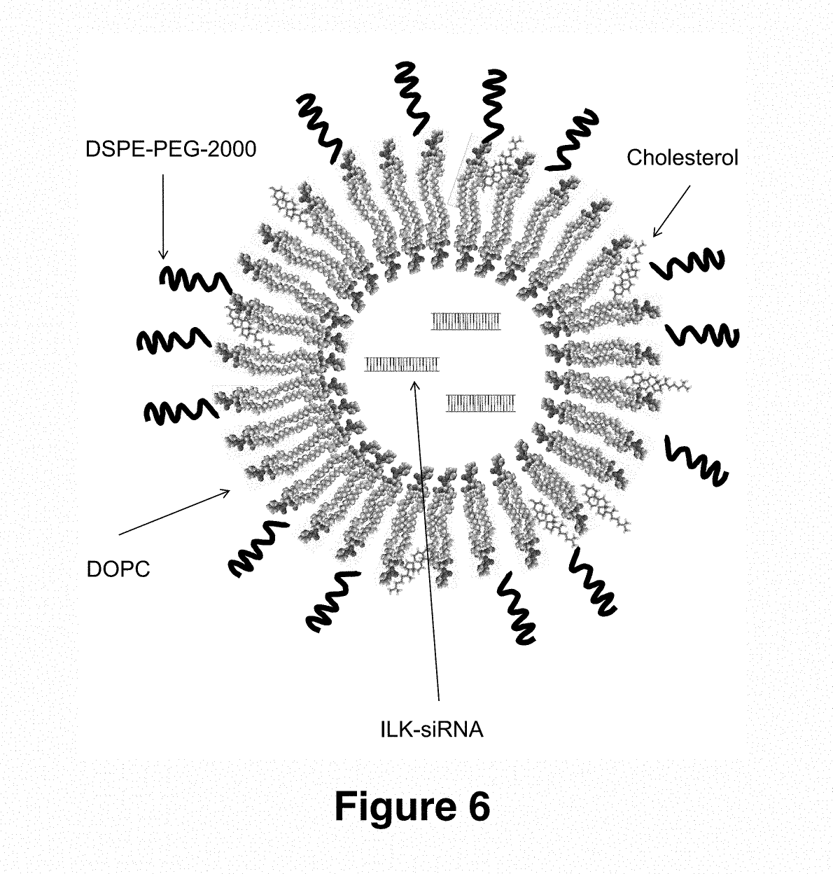

For in vivo delivery, siRNAs were mixed with DOPC, DSPE-PEG-2000 and cholesterol in the presence of excess terbutanol, frozen in an acetone-dry ice bath and lyophilized. Before in vivo administration, the lyophilized powder was hydrated with Ca²⁺ and Mg²⁺-free PBS at a concentration of 25 mg/mL to achieve the desired dose of 5 μg of siRNA in 200 μl/injection. FIG. 6 is a schematic representation of the nanoliposomal formulation of the present invention. The nanoliposomal formulation comprises a phospholipid, a lipid, a PEG-phospholipid and ILK-siRNA. In a preferred embodiment of the invention the nanoliposomal formulation comprises small interference RNA (siRNA) targeting ILK, 1,2-dioleoyl-sn-glycero-3-phosphatidylcholine (DOPC), 1,2-distearoyl-sn-glycero-3-phosphoethanolamine-N-[amino(polyethylene glycol) 2000](DSPE-PEG-2000), and cholesterol. Specifically, the nanoliposomal formulation comprises the lipids and ILK-siRNA according to the following ratios: siRNA:DOPC 1:10 w/w; DOPC:cholesterol 1:0.5 w/w; DSPE-PEG-2000:55 mol/mol DOPC. The preparation method for the formulation was described in copending U.S. application Ser. No. 14/681,875, incorporated in its entirety herein by reference.

### Tumor Implantation and In Vivo Treatment

Female athymic nude mice (NCr-nu, 6 weeks old) were purchased from Taconic. Briefly, to assess the efficacy of ILK silencing in vivo, three weeks after intraperitoneal (i.p.) injection with A2780CP20 cells (1×10⁶ cells/0.2 mL HBSS), mice were treated (i.p.) twice with C-siRNA-DOPC-PEG-nanoliposomes or ILK-siRNA-DOPC-PEG-nanoliposomes (5 μg siRNA per mice). The second injection was applied 2 days after the first one. Mice were euthanized one day after the last injection. The peritoneal tumors were harvested and tissue samples were snap-frozen in liquid nitrogen and stored at −80° C. for protein analysis by Western blot with a specific antibody against ILK. To evaluate the therapeutic activity of ILK siRNAs alone or in combination with cisplatin (CIS) in vivo, A2780CP20 cells were injected as described above. Seven days after tumor implantation, mice were randomly divided into the following treatment groups (N=10 per group): (a) C-siRNA, (b) ILK-siRNA, (c) C-siRNA plus CIS, and (d) ILK-siRNA plus CIS. DOPC-PEG-liposomal-siRNAs (5 μg siRNA/injection) and CIS (160 μg/injection) were injected i.p. once a week for 4 weeks. At the end of the treatment, mice were euthanized, the entire peritoneal cavity was examined for tumor metastases, and tumors were collected. The number of tumor nodules and tumor weight was recorded. Tumor specimens were fixed in formalin for paraffin slide preparation, or snap-frozen in liquid nitrogen for protein analysis.

### Statistical Analysis

In vitro experiments were performed between 3 to 5 times. For in vitro and in vivo experiments, statistical analysis was performed using Student's t-test for comparing two groups and by ANOVA for multiple group comparisons. P-values of <0.05 were consider statistically significant. GraphPad Prism software was used for graphing and statistical analysis.

### Results

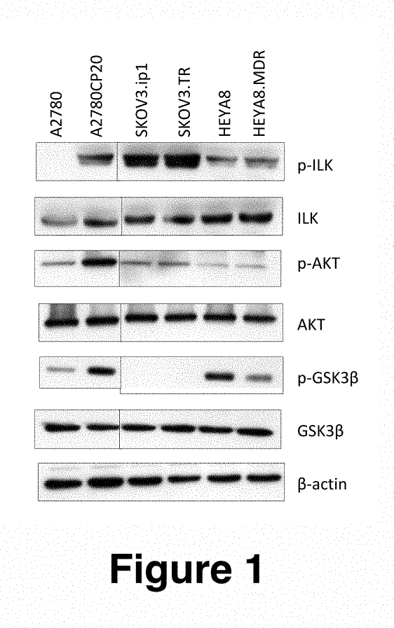

Expression of ILK downstream effectors in ovarian cancer cells Western blot analysis (FIG. 1A) of a panel ovarian cancer cell lines showed that phsphorylation levels of ILK (Ser246), AKT (S473) and GSK3b (Ser9) were higher in cisplatin-resistant A2780CP20 and OV90CIS compared with their parental counterparts A2780 and OV90, respectively.

### Effects of ILK Silencing on Cell Growth and on the Invasive Ability of Cisplatin Resistant Ovarian Cancer Cells

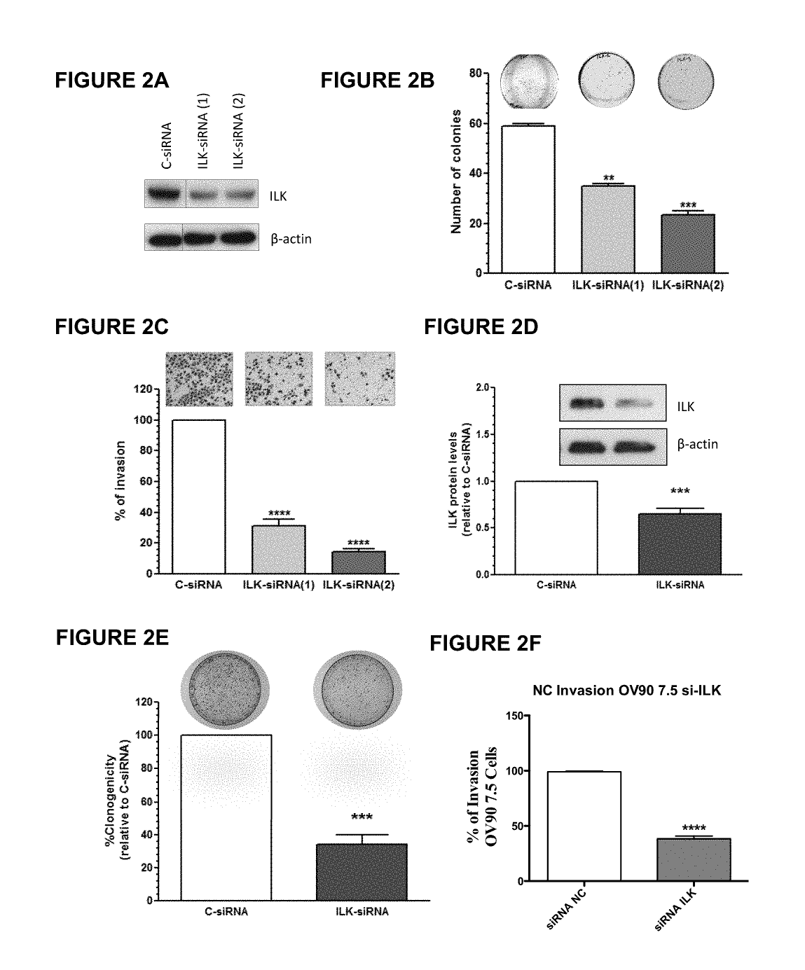

Next, we studied whether silencing ILK reduced cell growth and in the invasion of cisplatin resistant ovarian cancer cells. Transient transfection of ILK-targeted siRNAs into A2780CP20 cells significantly decreased the ILK protein levels compared with the C-siRNA (FIG. 2A). In a colony formation assay, both ILK-targeted siRNAs reduced number of colonies formed compared with the C-siRNA transfected cells (FIG. 2B). Particularly, the ILK-siRNA(2) reduced in 76.7% (***p<0.001) the number of colonies compared with the C-siRNA (FIG. 2B), whereas ILK-siRNA(1) reduced the number of colonies in only 65% (**p<0.01). (FIG. 2B).

Increased levels of ILK have been associated with accelerated metastatic rate of ovarian cancer and other tumors. Thus, we assessed whether ILK silencing was able to reduce the invasive ability of cisplatin-resistant ovarian cancer cells. Invasion assays confirmed that ILK-siRNA(1) and ILK-siRNA(2) significantly reduced (68.7% reduction; ****p<0.0001 and 85.5% reduction; ****p<0.001, respectively) the invasiveness of A2780CP20 cells compared with C-siRNA transfected cells (FIG. 2C). Colony formation and invasion assays were repeated in the high-grade serous ovarian cancer (HGSOC) cells, OV90CIS. Transient transfection of the ILK-siRNA(2) into these cells reduced significantly the ILK protein levels in these cells (FIG. 2D). Under these conditions, the number of colonies were reduced in 65.8% (***p<0.001) (FIG. 2E), and the number of invaded cells were reduced in 61.6% (****p<0.0001) (FIG. 2F).

Effect of ILK Silencing in Combination with Cisplatin on Cell Viability

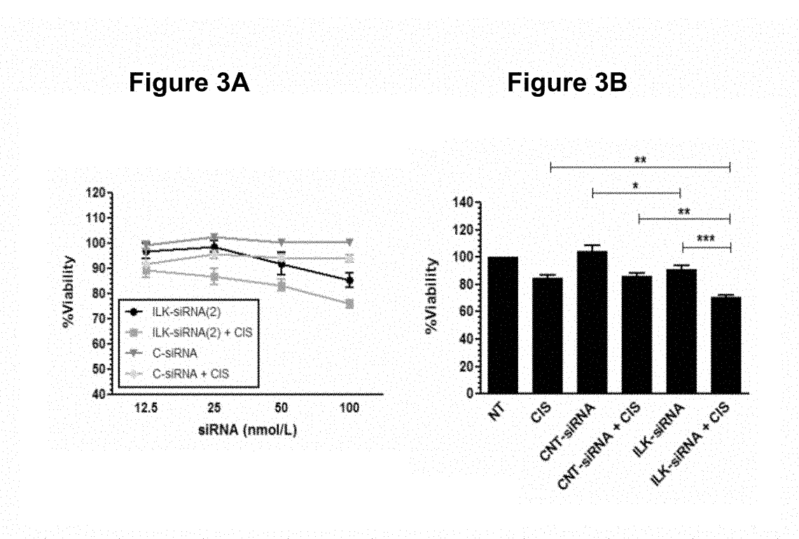

Then we investigated if ILK-targeted siRNAs alone or in combination with cisplatin induced effects in cell viability. Transient transection of 50 nM and 100 nM of ILK-siRNA(2) in A2780CP20 cells reduced significantly the cell viability compared with the C-siRNA (FIG. 3A). Combination of ILK-siRNA(2) with inactive doses of cispaltin (2 μM) significantly reduced cell viability even at siRNA doses as low as 25 nM (final concentration) (FIG. 3A-B).

Therapeutic Effect of DOPC-PEG-Liposomal siRNAs

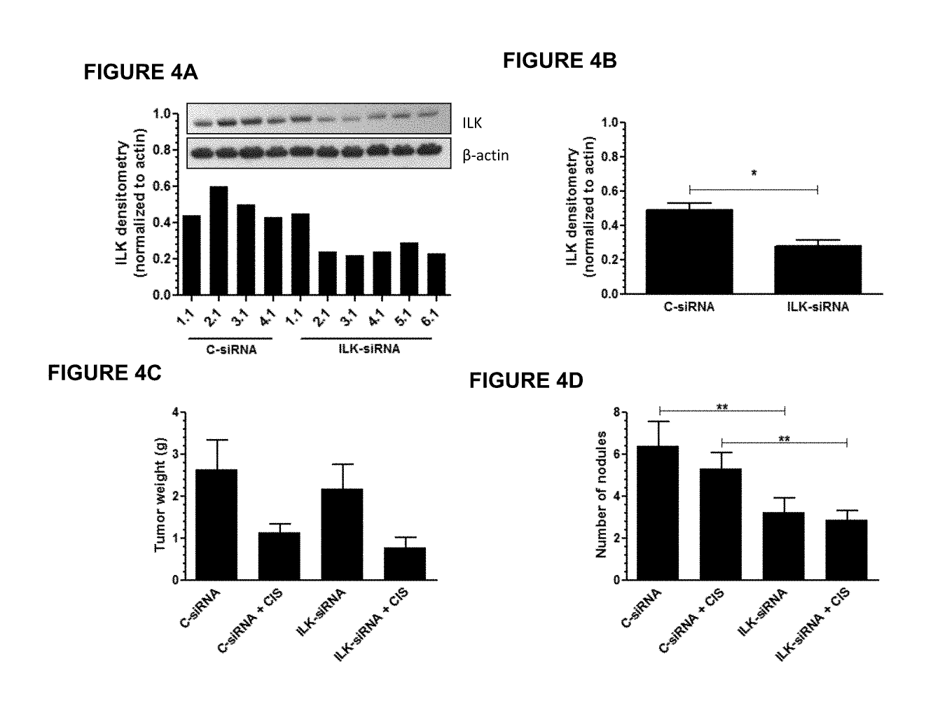

We then investigated if a nanoliposomal formulation of ILK-siRNA(2) reduced in vivo tumor growth. Characterizations of these liposomes have been reported previously. First, we performed an experiment to demonstrate that the liposomal ILK-siRNA(2) reached the tumor and decreased ILK protein levels on it. Three weeks after A2780CP20 cell implantation, mice were injected with two doses of ILK-siRNA(2). The second injection was administered two days after the first one. Western blot analysis in protein extracted from tumor tissues, showed that the liposomal-siRNA(2) visibly decreased the ILK protein levels in all mice compared with the liposomal-C-siRNA (FIG. 4A). Densitometric analysis of the intensity bands confirmed that the liposomal-ILK-siRNA(2) significantly decreased the ILK protein levels (*p<0.05) compared liposomal-C-siRNA group (FIG. 4B).

In therapeutic experiments, the liposomal-ILK-siRNA(2) did not reduced significantly the tumor weight (FIG. 4C). However, the liposomal formulation decreased the number of tumor nodules (**p<0.01) compared to C-siRNA treated mice (FIG. 4D). In the combination group, ILK-siRNA(2) plus cisplatin reduced significantly the number of tumor nodules compared with the C-siRNA plus cisplatin group (FIG. 4D).

### Effect of ILK Silencing on its Downstream Effectors

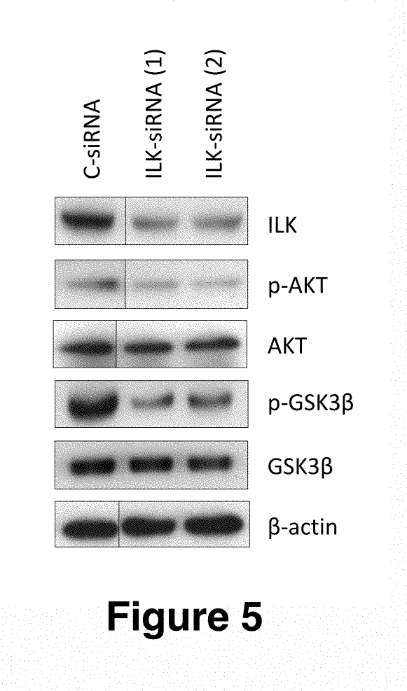

Reports indicate that phosphorylation of ILK activate (phosphorylate) AKT and GSK3b. Phosphorylation of AKT activates mTOR and mTOR downstream effectors. On the other hand, phosphorylation in Ser-9 of GSK3b inactive it, which is enable to phosphorylate b-catenin. Phosphorylated b-catenin is accumulated in the cytoplasm with subsequent translocation into the nucleus to acts as a co-activator of transcription. However the precise ILK downstream effectors contributing to the cisplatin resistance of ovarian cancer cells have not been completely understood. Western blot analysis shows that transient transfection in A2780CP20 cells with ILK-targeted siRNAs [ILK-siRNA(1) and ILK-siRNA(2)] significantly reduced ILK protein levels (FIG. 5). Decrease in the ILK protein levels resulted in a significant reduction in p-AKT and p-GSK3b protein levels (FIG. 2A). However the total forms of AKT and p-GSK3b remains unchanged (FIG. 5).

### DISCUSSION

One important aspect of this invention is that siRNA-mediated IILK silencing reduces the phosphorylation but not the total levels of its downstream effectors AKT and GSK3b. Evidence indicates that post-translational modification of proteins plays a central role in drug resistance. Altered protein phosphorylation levels play a central role in signaling pathways controlling cell growth, proliferation and metastasis of cancer cells.

Increased levels of ILK have been well documented in various types of cancer. Hwang and co-workers showed that tumor growth factor (TGFβ1) activates ILK and stimulates invasion and migration of the SKOV3 human ovarian cancer cell line. Similarly, Cho and co-workers showed that ILK-targeted siRNA abrogated the invasive potential of ovarian cancer cells and reduced in vivo tumor growth.

ILK phosphorylates AKT, GSK3β, and other substrates. AKT is a serine/threonine kinase that regulates various cell survival pathways. AKT is phosphorylated at high levels in various human tumor types, and a subset of ovarian cancers and cells in culture. Tsang and co-workers showed that AKT confers cisplatin resistance through the inhibition of the Fas-associated death domain-like interleukin-1β-converting enzyme (FLICE)-like inhibitory protein (FLIP)/p53 pathway, which activates apoptosis. Asselin and co-workers also showed that siRNA-mediated targeting of AKT sensitized endometrial carcinoma cells to cisplatin induced apoptosis.

The present invention shows additional evidence that phosphorylated AKT plays an important role in cisplatin resistance, because siRNA-mediated ILK silencing reduced p-AKT protein levels but not total AKT levels. Additionally, p-AKT levels, but not total AKT levels, were higher in A2780CP20 and OV90Cis cisplatin-resistant cells compared with their respective parental counterparts.

The phosphorylated GSK3β levels also decreased following ILK silencing in cisplatin-resistant cells. In addition to glycogen synthesis, the GSK3β isoform phosphorylates a number of substrates including metabolic and signaling proteins, structural proteins and transcription factors that regulate cell survival. Evidence demonstrates that GSK3β is involved in the process of tumorigenesis. Phosphorylation of human GSK3β at tyrosine 216 is critical for efficient kinase activity, whereas GSK3β kinase activity is inhibited through phosphorylation of serine 9 by AKT and other kinases. Reports have shown that inhibition of the expression and activity of GSK3β in SKOV3 cells attenuated cell proliferation, caused apoptosis, and reduced in vivo tumor growth.

Overall, the present invention provides further evidence that phosphorylation levels of ILK/AKT/GSK3b pathway contributes to the cisplatin-resistant phenotype of ovarian cancer cells and that targeting ILK with siRNA is a therapy for cisplatin resistant ovarian cancer.

Although the present invention has been described herein with reference to the foregoing exemplary embodiment, this embodiment does not serve to limit the scope of the present invention. Accordingly, those skilled in the art to which the present invention pertains will appreciate that various modifications are possible, without departing from the technical spirit of the present invention.

1. A nanoliposomal formulation for treating cancer cells in mammals comprising: ILK-siRNA, a phospholipid, a lipid and a PEG-phospholipid.

2. The nanoliposomal formulation of claim 1, wherein said phospholipid is 1,2-dioleoyl-sn-glycero-3-phosphatidylcholine (DOPC), said PEG-phospholipid is 1,2-distearoyl-sn-glycero-3-phosphoethanolamine-N-[amino(polyethylene glycol)-2000] (DSPE-PEG-2000), and said lipid is cholesterol.

3. The nanoliposomal formulation of claim 2, wherein the concentration for said formulation comprises the ratios: siRNA:DOPC 1:10 w/w; DOPC:cholesterol 1:0.5 w/w; and DSPE-PEG-2000:55 mol/mol DOPC.

4. The nanoliposomal formulation of claim 1, wherein said cancer cells are ovarian cancer cells.

5. A method of reducing cancerous cell proliferation and invasion in mammals, said method comprising: targeting Integrin Linked Kinase (ILK) with a nanoliposomal formulation composed of ILK-siRNA, a phospholipid, a lipid and a PEG-phospholipid.

6. The method of claim 5, wherein said phospholipid is 1,2-dioleoyl-sn-glycero-3-phosphatidylcholine (DOPC), said PEG-phospholipid is 1,2-distearoyl-sn-glycero-3-phosphoethanolamine-N-[amino(polyethylene glycol)-2000] (DSPE-PEG-2000), and said lipid is cholesterol.

7. The method of claim 6, wherein the concentration for said formulation comprises the ratios: siRNA:DOPC 1:10 w/w; DOPC:cholesterol 1:0.5 w/w; and DSPE-PEG-2000:55 mol/mol DOPC.

8. The method of claim 5, wherein said cancerous cells are ovarian cancer cells.
